(19) United States
(12) Patent Application Publication
(10) Pub. No.: US 2009/0270819 A1
(43) Pub. Date: Oct. 29, 2009

(54) OPTICAL SAFETY TROCAR AND METHOD OF USE THEREOF

(76) Inventors: Dario Vitali, New Smyma Beach, FL (US); Rogelio Insignares, Miami, FL (US); Ken Roger, Casselbury, FL (US); Clay Fette, Palm Beach Gardens, FL (US)

Correspondence Address: Innovative IP, LLC, P.O. Box 217, New Smyrna Beach, FL, 32169, US

(21) Appl. No.: 12/111,625

(22) Filed: Apr. 29, 2008

(51) Int. Cl. A61M 5/158

(52) U.S. Cl. 604/272

(57) ABSTRACT

An optical safety trocar and method of use thereof, wherein a shield deploys to guard against accidental tip contact and potential injury therefrom, and wherein an optical tip facilitates visualization of the trocar advancement into a body cavity, such that the visualization not only precludes blind penetration in order to avoid inadvertently injurious positioning of the tip, but enables identification of any unintentional and otherwise undetectable damage.

### FIELD OF THE INVENTION

The present invention relates generally to trocars, and more specifically, to an optical safety trocar and method of use thereof, wherein a shield deploys to guard against accidental tip contact and potential injury therefrom, and wherein an optical tip facilitates visualization of the trocar advancement into a body cavity, such that the visualization not only precludes blind penetration in order to avoid inadvertently injurious positioning of the tip, but enables identification of any unintentional and otherwise undetectable damage.

### BACKGROUND OF THE INVENTION

Minimally invasive surgery is currently well established for numerous surgical applications, such as laparoscopy and arthroscopy. In such procedures, small incisions replace those previously required for open surgery, wherein medical instruments and miniaturized visualization tools are instead operated through access ports that pass through the skin and tissue into body cavities. The insertion of the port is typically accomplished via the use of a trocar, generally comprising an obturator and a cannula.

In use, the obturator essentially defines the incision point and creates a passageway through which the cannula enters the body cavity. Thus, and in general, a bladeless trocar serves as a temporary housing for an obturator, wherein the bladed obturator creates the entryway for the trocar cannula to pass through and into the body, and wherein the obturator is withdrawn to enable use of the trocar cannula as an accessway into the body cavity for application of surgical procedures. For example, the trocar cannula commonly performs as a port of entry for surgical instruments such as a clip applier, scissors, retractor, and/or endoscope for surgical procedures involving cutting, fastening, coagulating and/or excising tissue.

Endoscopic surgery is thus one such application, wherein an endoscope is inserted through a cannula for visualization within the body. There are currently a variety of trocars available for use in endoscopic surgery. Pyramidal trocars, blunt obturator trocars, bladeless trocars, and shielded trocars are the most common. Blunt obturator, or blunt tip trocars are intended to minimize the likelihood of inadvertent internal injury, wherein rather than creating an incision through intact skin, these devices rely upon punctures created by a needle during insufflation, a procedure that commonly precedes endoscopic surgery, and serve to enlarge the preexisting puncture openings for cannula insertion therethrough. Such devices are disadvantageous in view of the present invention, wherein puncture wounds have been found to have slower healing with increased risk of complications when compared with small razor incisions, and wherein visual determination of the forward position of the advancing trocar is limited to depth markings on the cannula exterior.

Rather than utilizing needle punctures, a sharp trocar obturator or stylet creates an incision to enable passage of a cannula. Because pressure must be exerted at the incision site in order to penetrate the skin and tissue, the force of entry of the trocar into the body cavity can potentially result in internal injury. Therefore, shields have been designed to essentially cover the sharp or bladed portion while blind entry into a body cavity is accomplished, in order to prevent inadvertent contact with and accidental injury to internal organs or the like.

Some available shields require proactive deployment by the physician, wherein the necessary manipulation of the trocar for shield deployment can lead to disruption of the preferred orientation of the trocar. Other shields are essentially self-deployed, prior to and after insertion into and passage through the body wall, respectively, during insertion. Most shields have a tubular configuration in order to surround the cutting end of the trocar; however, activation or deployment of many of these shields within the body cavity can be inhibited or at least disadvantageously slowed by tissues immediately surrounding the trocar, such that protection is only realized from shield deployment occurring only after passage of the entire tubular shield circumference through the skin tissue and into the body cavity. In such cases, inadvertent internal injury can occur during entry and before the shield can deploy fully. Moreover, increased resistance at the point of insertion from such a shield can necessitate delivery of increased force in order for penetration to occur. Such an increase in force can result in a further risk of injury immediately following insertion. Further, some shields disadvantageously require an increase in incision dimensions, with attendant increased risk of wound infection and healing time.

Spring-loaded tubular shields have been described and provided, wherein after entry into the body cavity is enabled by the obturator via creation of a suitably and minimally sized opening, the shield springs into the body cavity, surrounds the sharp end of the obturator, and prevents accidental contact with internal organs. One such exemplary device is the subject of U.S. Pat. No. 7,153,319B1 to Haberland et al. It is noted that although using a trocar with such a shield does mitigate the risk associated with insertion, there remains a chance of accidental injury to internal tissues. Moreover, an accidental injury can remain unnoticed during surgery due to the lack of a visualization method during insertion.

Another approach to reducing accidental injury has been through the use of a transparent tip with a blade edge. Devices with such tips are intended to facilitate telescopic video or direct eyepiece viewing of the insertion process in order to enable monitoring of the incision and entry in order to avoid accidental injury, wherein the surgeon can view the tissue surrounding the transparent tip during penetration. The monitoring is facilitated by utilization of an endoscope, a miniaturized camera, or elongated imaging and visualization instrument. The images are typically transmitted to a monitor screen for viewing by the surgical team. These instruments thus enable visibility of the tissue during the advancement of the obturator. Most of the transparent tip designs are generally intended to minimize the visual obstruction realized from the cutting member; nevertheless, a line or narrow band from the blade typically blocks the central portion of the visualization range.

Transparent blades have thus been proposed in order to avoid obstruction of the field of view. However, while the objective of transparency may be achieved, the blade characteristics necessarily limit the materials that may be selected for use in manufacture, and an external, removable cap is required for the protection of the tip during periods of nonuse, such that removal thereof would be necessarily required to facilitate use. Moreover, even during full view, accidental movement and contact within internal tissues may occur, wherein no automatic protective features are offered for quality assurance in the face of human oversight, accident, or error.

Additionally, such designs do not provide protection for a surgeon from inadvertent contact with the tip. One attempt to overcome such disadvantage has been to render the cutting member retractable; however, such configurations disadvantageously either rely on overt action by the surgeon for both the engagement and retraction of the blade, or include a default retraction setting that is unrelated to the position of the trocar relative to the body cavity, but is instead directed according to mechanism settings selected by the user.

Therefore, it is readily apparent that there is a need for a trocar that enables visualization during entry, but that also functions as a true safety trocar, thereby avoiding the above-discussed disadvantages.

### BRIEF SUMMARY OF THE INVENTION

Briefly described, in a preferred embodiment, the present invention overcomes the above-mentioned disadvantages and meets the recognized need for such a device by providing a safety trocar with an optical tip that allows visualization of entry in combination with a shield that deploys upon entry to protect internal tissues from accidental damage, wherein the use of entry visualization in combination with a shield serves to reduce the risk of injury while coincidentally increasing the likelihood of identification of any accidental injury that may occur.

According to its major aspects and broadly stated, in its preferred form, the present invention is a method of decreasing risk of accidental injury during minimally invasive surgical procedures such as, for example, endoscopy, arthroscopy, or laparoscopy, and also of decreasing risk of complications from unnoticed injuries by utilizing a trocar comprising a safety shield and an optical tip.

More specifically, the device of the present invention in its preferred form comprises an optically tipped and shielded trocar. The preferred combination of features serves to facilitate entry into the body for surgery via a minimal incision, while also maximizing safety. That is, the shield functions to protect the internal tissue from accidental injury during insertion, while the optical tip functions to allow for the use of a visualization method during insertion. The beneficial addition of a visualization method to a true safety trocar allows the end-user to monitor the progress of trocar insertion, thereby beneficially reducing the risk of accidental internal tissue damage even further than previously described devices. The ability to monitor entry and view the status of surrounding tissues both while the shield is deployed and also while the shield is retracted further serves to increase the likelihood that a member of the surgical team will be able to identify if and/or when accidental internal tissue damage occurs so that it can be timely repaired and unnecessary complications can be avoided.

The shield is preferably at rest in a deployed or extended position such that the necessity for a removable protective cover for the cutting tip is alleviated, yet members of the surgical team as well as the patient are protected from accidental injury both before and after respective entry and withdrawal of the obturator into and from the patient. Additionally, the blade itself is protected from damage prior to use, again without the need for a removable protective cap or covering. This preferred design thus eliminates the need to remove and/or keep track of a small cap within the operatory theatre.

The shield is pushed back away from the tip and exposing the blade for cutting during insertion. Thereafter, the shield springedly re-deploys, extending past the blade tip. The preferred sloped design of the trocar tip facilitates passage of the shield through the surgical incision, and thus enables the realization of protective benefits from the shield even before the entire trocar tip has entered the body cavity, and with a minimally dimensioned incision site. That is, the strategic positioning of the blade within the angled trocar tip, and with the shield proximate thereto, enables the blade to create an incision and enables a first portion of the shield to deploy through the incision, defining a protective safety barrier proximate the blade without necessitating full passage of the circumference of the shield into the body cavity.

The preferred trocar of the present invention also has an inner lumen or channel that allows for the passage of an endoscope within the length thereof, and down to the tip, wherein the tip is preferably optically clear. This configuration facilitates visualization through the tip during insertion in order to observe the incision procedure and also in order to confirm shield deployment upon entry into the body cavity.

Thus, a feature and advantage of the present invention is its ability to provide a method for achieving visualized entry into a body with a shielded obturator.

Another feature and advantage of the present invention is its ability to provide a method for reducing the risk of accidental injury during surgery through the use of an optically tipped trocar with a safety shield.

Another feature and advantage of the present invention is its ability to provide a trocar comprising both an optical tip and a safety shield.

Yet another feature and advantage of the present invention is its ability to guard against accidental tip contact and potential injury therefrom while coincidentally precluding blind penetration and enabling identification of any unintentional and otherwise undetectable damage.

Still another feature and advantage of the present invention is its ability to safely define a surgical access port through tissue and into a body cavity.

Yet another feature and advantage of the present invention is its ability to facilitate visual determination of the forward position of the tip of during advancement.

Still another feature and advantage of the present invention is its ability to deploy a protective shield without necessitating proactive deployment action or manipulation by the operator.

Yet still another feature and advantage of the present invention is its ability to avoid inhibition of shield deployment by tissues proximate the trocar.

Still yet another feature and advantage of the present invention is its ability to provide a complete field of view without necessitating limitations in blade materials and opacity.

Still yet another feature and advantage of the present invention is its ability to provide automatic protective features for quality assurance in the face of human oversight, accident, and/or error.

Still another feature and advantage of the present invention is its ability to visualization during entry, but to also function as a true safety trocar.

And, still another feature and advantage of the present invention is its ability to provide for using, assembling, and positioning a surgical device, and for viewing therethrough, via incorporation of an at least optically translucent tip portion onto a tubular trocar.

These and other objects, features and advantages of the present invention will become more apparent to one skilled in the art from the following description and claims when read in light of the accompanying drawings.

### BRIEF DESCRIPTION OF THE DRAWINGS

The present invention will be better understood by reading the Detailed Description of the Preferred and Alternate Embodiments with reference to the accompanying drawing figures, in which like reference numerals denote similar structure and refer to like elements throughout, and in which:

FIG. 1A is a cross-sectional view of an optical safety trocar obturator according to a preferred embodiment of the present invention;

FIG. 1B is an enlarged, partial view of the obturator of FIG. 1A;

FIG. 2A is a partial, cross-sectional view of an optical safety trocar according to a preferred embodiment of the present invention, showing the shield in a deployed position against skin S, according to a first step of the preferred method of use;

FIG. 2B is a partial, cross-sectional view of an optical safety trocar according to a preferred embodiment of the present invention, showing the shield in a partially retracted position and showing the blade against skin S, according to a second step of the preferred method of use;

FIG. 2C is a partial, cross-sectional view of an optical safety trocar according to a preferred embodiment of the present invention, showing the shield in a fully retracted position and showing the blade partially cut through skin S and tissue T, according to a third step of the preferred method of use;

FIG. 2D is a partial, cross-sectional view of an optical safety trocar according to a preferred embodiment of the present invention, showing the shield in a deployed position within body cavity C, according to a fourth step of the preferred method of use;

FIG. 3A is a perspective view of an optical safety trocar according to a preferred embodiment of the present invention, showing the shield in a deployed position, extending past the blade and proximate an exterior body surface;

FIG. 3B is a perspective view of an optical safety trocar according to a preferred embodiment of the present invention, showing the shield in a partially retracted position, extending essentially equidistant with the blade, with both proximate an exterior body surface;

FIG. 4A is a partial, cutaway, perspective view of an optical safety trocar according to a preferred embodiment of the present invention, showing the shield in a fully retracted position, with the blade extending outwardly therefrom, with the blade partially cut through an exterior body surface;

FIG. 4B is a partial, cutaway, perspective view of an optical safety trocar according to a preferred embodiment of the present invention, showing the shield in deployed position, extending past the blade within the interior of the body;

FIG. 5A is a cross-sectional view of a trocar assembly according to an alternate configuration with a polymer tip, showing a trocar obturator and cannula assembly with a palm handle;

FIG. 5B is an enlarged, partial view of the trocar assembly of FIG. 5A, showing the polymer tip, shield, and blade;

FIG. 5C is an enlarged, partial view of the trocar assembly of FIG. 5A, showing the shield mechanism;

FIG. 6 is a side perspective view of the trocar assembly of FIG. 5A;

FIG. 7A is a cross-sectional view of a trocar assembly according to an alternate embodiment of the present invention, showing a trocar obturator and cannula assembly with a pistol grip;

FIG. 7B is an enlarged, partial view of the trocar assembly of FIG. 7A, showing the optical tip and shield;

FIG. 7C is an enlarged, partial view of the trocar assembly of FIG. 7A, showing the shield mechanism;

FIG. 8 is a side perspective view of the trocar assembly of FIG. 7A;

FIG. 9A is a cross-sectional view of a trocar assembly, showing a scope inserted therewithin; and

FIG. 9B is a partial, perspective view of an exemplary scope for use in combination with the present invention.

### DETAILED DESCRIPTION OF THE PREFERRED AND ALTERNATE EMBODIMENTS

In describing the preferred and alternate embodiments of the present invention, as illustrated in the figures and/or described herein, specific terminology is employed for the sake of clarity. The invention, however, is not intended to be limited to the specific terminology so selected, and it is to be understood that each specific element includes all technical equivalents that operate in a similar manner to accomplish similar functions.

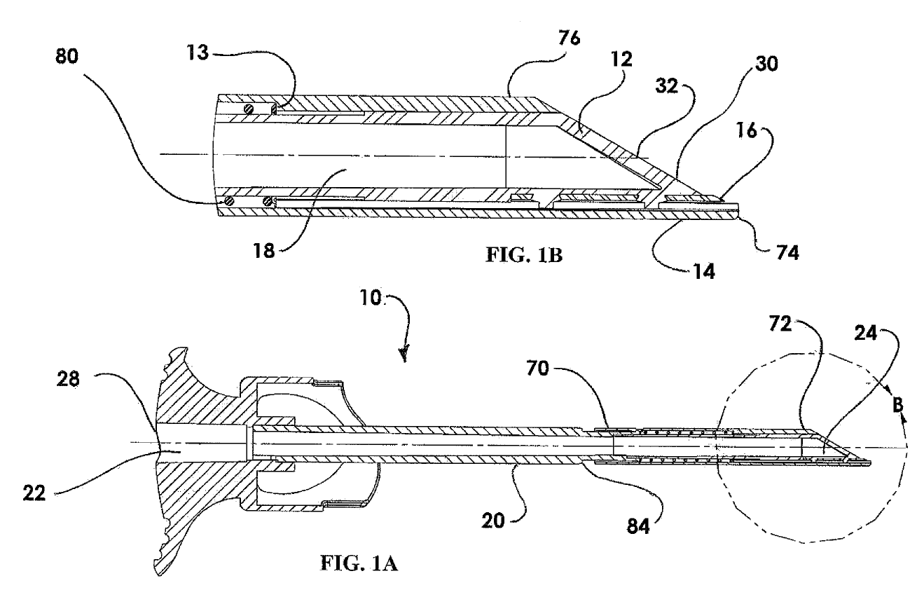

Referring now to FIGS. 1A-4B, the present invention in the preferred embodiment is optical safety trocar 10, generally comprising optical tip 12, shield 14, blade 16, and channel 18 to define obturator 20 for use with a cannula. Obturator 20 is an elongate, cylindrically-shaped tool, wherein channel 18 is defined therewithin, extending from proximal end 22 to distal end 24.

Channel 18 is preferably dimensioned to accommodate passage of visualization equipment therewithin, such as, for exemplary purposes only, an endoscope, fiber optics, laser, and/or other lighting and/or electric signal coupling elements, in order to enable visual image and/or video transmission therethrough. One skilled in the art should readily recognize that although an endoscope is typically the preferred visualization tool for utilization with trocars, any suitable visualization aid may be utilized without departing from the intended scope of the present invention, including an eyepiece. Port 28 is preferably defined at proximal end 22 of channel 18 for passage of visualization equipment therethrough. Distal end 24 of channel 18 is defined proximate optical tip 12, wherein optical tip 12 functions to allow collection of visual images or video therethrough by the selected visualization equipment, preferably an endoscope.

The flat surface 32 of distal end 24 of optical tip 12 minimizes and/or eliminates image distortion therethrough, wherein optical tip 12 is preferably essentially transparent in order to allow visualization and/or transmission of an image therethrough, with materials preferably comprising a thermoplastic, and may further comprise, without limitation, acrylic, polycarbonate, polyester, polymethylmethacrylate (PMMA), polymethylpentene, polypropylene, polysulfones, cellulose acetate, styrene acrylic co-polymers, fluoropolymers, polystyrene, polyetherimides, polyurethane, styrene acrylonitrile, silicones, epoxys, polyvinyl chloride, urethane, acrylonitrile-butadienestyrene (ABS) allyl diglycolocobane, co-polyesters, transparent epoxies, glass and/or any other suitable material or combination thereof with the necessary visualization characteristics.

Blade 16 is preferably positioned proximate shield 14, such that when shield 14 is at rest, shield 14 extends outwardly from optical tip 12 a greater distance than blade 16. Further, when shield 14 is retracted during incision, as will be further discussed hereinbelow, shield 14 no longer extends outwardly from optical tip 12, wherein blade 16 is generally exposed for use. This preferred positioning for blade 16 facilitates maximized visualization through optical tip 12, wherein blade 16 does not cross, divide or otherwise interrupt the field of view.

Preferably, blade 16 is a small, flat razor-type blade configured to allow insertion into the body with minimal tissue trauma. One skilled in the art would recognize, however, that although such a blade is preferable, other cutting tips could alternately be used, such as cone or pyramid cutting tips. Further, any desirable material or combination thereof may be utilized for manufacture of blade portion, such as, without limitation, metal, ceramic, plastic, and/or glass. The beneficial asymmetric geometrical configuration of tip 12 and blade 16 enable a minimization of the wound via creation of a slit-like incision, wherein passage of the angular trocar obturator 20 is enabled therethrough via smooth and gentle expansion or dilation of the tissue surrounding the incision and not via puncture of an incision of a diameter larger than trocar 10. That is, the sloped, angular shape of distal end 30 of optical safety trocar 10 facilitates passage of shield 14 through the surgical incision, and thus realization of protective benefits therefrom, even before the entire trocar shield 14 has entered the body cavity, and with a minimally dimensioned incision site.

Shield 14 is generally tubular-shaped, with proximal end 70 and distal end 72, wherein proximal end 70 is essentially flat, and wherein distal end 72 is essentially angled. That is, as shown at least in FIGS. 1A-1B, on cross-section, it can be observed that a first half of shield 14 proximate distal end 72 has a length 74 that exceeds length 76 of a second half of shield 14 in order to surround the preferred angular optical tip 12 and blade 16, as shown. Biasing forces for shield 14 are provided by spring 80, preferably surrounding the exterior surface of channel 18, and preferably extending from proximal end 13 of optical tip 12 to locking region 82 of shield 14.

With preferred similarity to shield configurations commonly owned and described by Haberland et al. in U.S. Pat. No. 7,153,319B1, shield stop 84 is preferably defined via a circumference with a measurement adequate to prevent further slidable retractive movement of shield 14 along the length of obturator 20. Alternately, lock mechanism 86 may further be included for shield 14, wherein snap finger 86 locks onto trocar 10 to secure shield 14 in a withdrawn position relative optical tip 12. It should be understood that further shield stop or lock features could also be incorporated into optical safety trocar 10, whereby shield 14 could be locked in a plurality of positions relative to optical tip 12. However, it is the preferred embodiment of the present invention that shield 14 is biased in a deployed position, that temporary retraction thereof is achieved via delivery of pressure force against a body, wherein blade 16 is exposed in order to create an incision, and that shield 14 automatically redeploys into the incision and into the body cavity.

If desired, coatings may be applied on shield 14, such as hydrophilic coatings, or shield 14 may be manufactured from hydrophilic components, in order to reduce friction and enhance speed of shield deployment via ease of entry through the body wall, especially when wetted. Exemplary non-reactive coatings that can reduce the coefficient of friction could include polyvinylpyrolidone-polyurethane, thermoplastic polyurethane or the like.

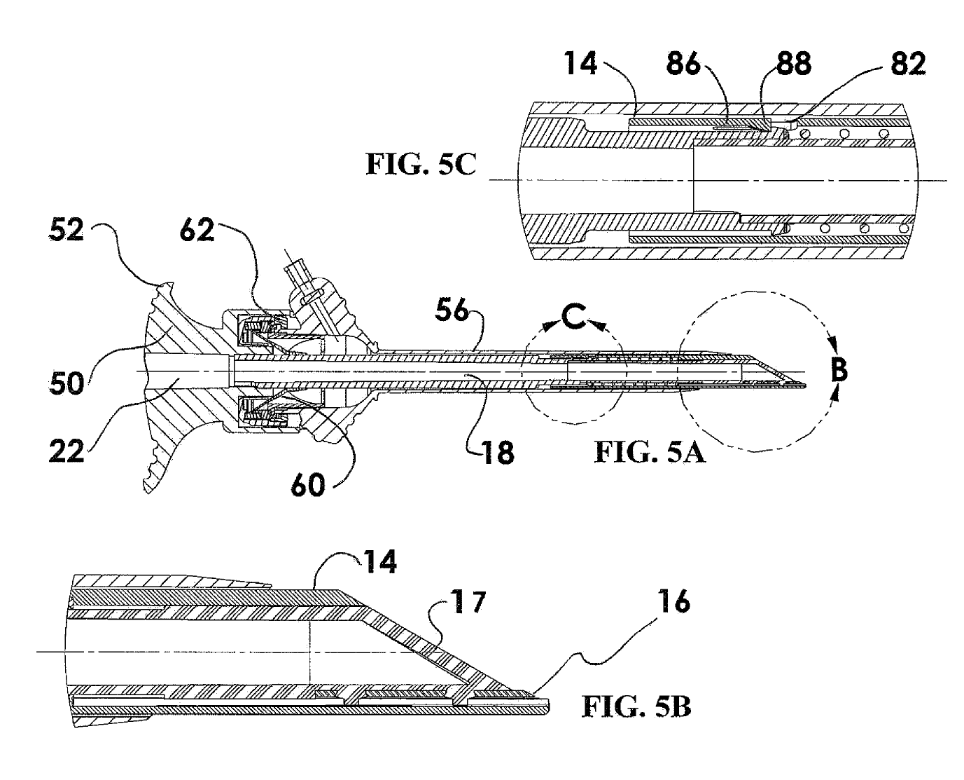

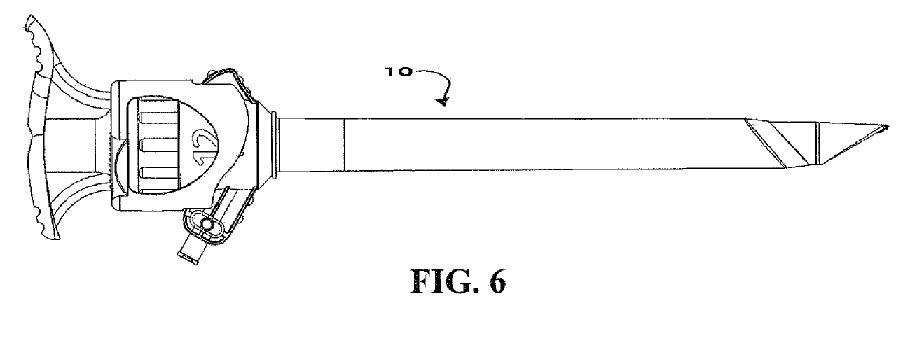

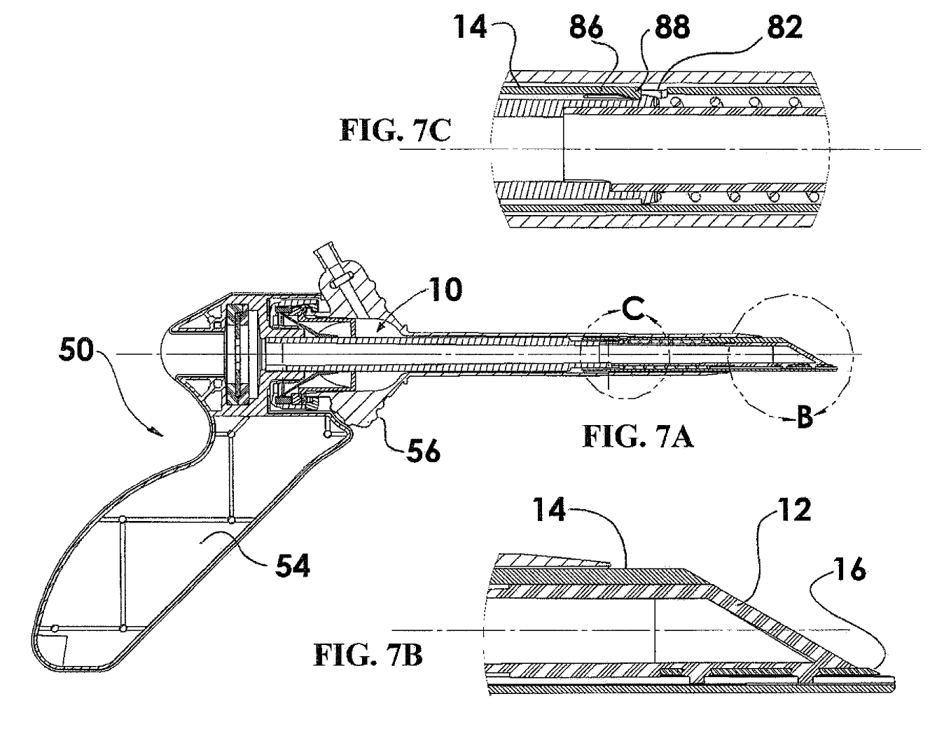

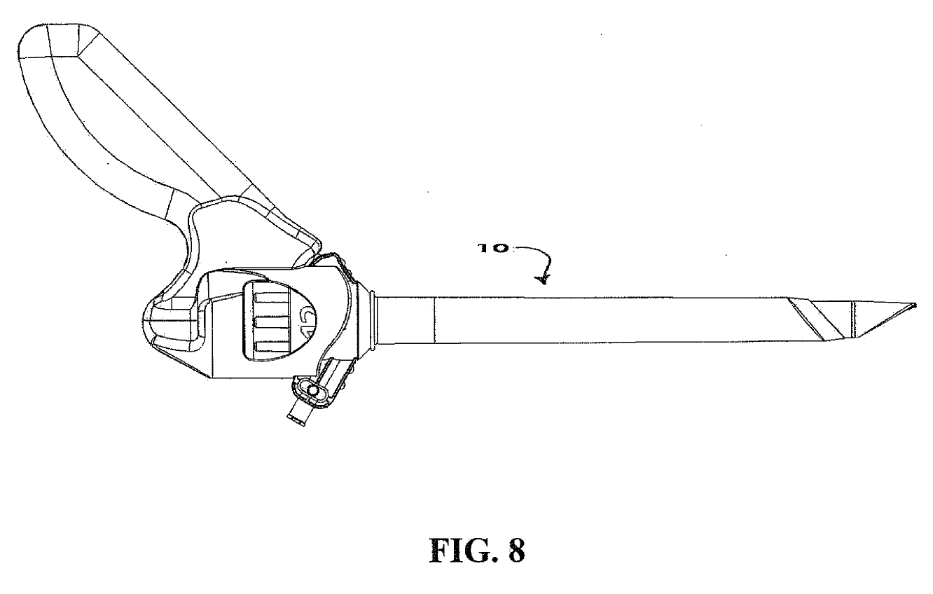

Referring now to FIGS. 6, 7A, and 8, optical safety trocar 10 further comprises handle 50, wherein handle 50 may be configured as palm handle 52, pistol grip handle 54, or any suitable handle adapted for integration with optical safety trocar 10. FIGS. 5A-5C are representative of the preferred functional configuration for shield 14 and blade 16 relative to trocar 10, but depict a polymer tip according to an alternate embodiment, rather than optical tip 12 Handle 50 preferably connects with cannula assembly 56, wherein utilization of optical safety trocar 10 therewith preferably maintains access to port 28 at proximal end 22 of channel 18 for passage of visualization equipment, such as an endoscope, therethrough during insertion into a body. Cannula assembly 56 is preferably slidably related with optical safety trocar 10, as further described by Haberland et al. U.S. Pat. No. 6,569,119 and related applications.

According to previously described and commonly owned improvements, such as described in pending U.S. non-provisional patent application Ser. No. 11/824,602, instrument seal 60 and annular retaining rings 62 are preferably defined proximate proximal end 22 of channel 18. Further detailed description regarding the configuration of this preferred instrument seal 60 is not provided herein, as to avoid repetition to previous disclosures, and also as to avoid limitation, wherein the improvements disclosed according to the present invention relate to optical tip 12 and shield 16, such that utilization of another seal design, shape, and/or configuration remains within the scope of the present invention.

In an alternate embodiment of the present invention, optical safety trocar 10 could be configured with a bladeless tip, wherein optical tip 12 could be suitably sharpened.

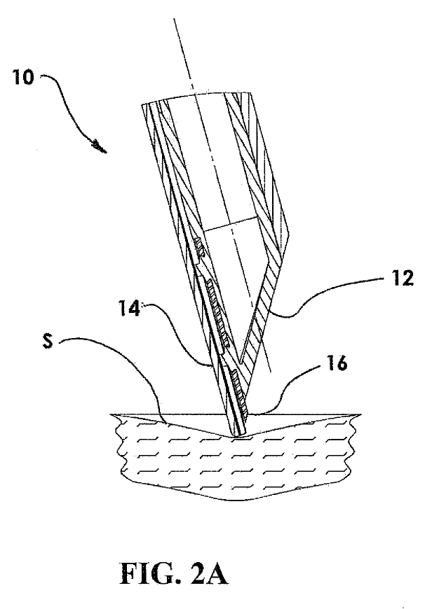

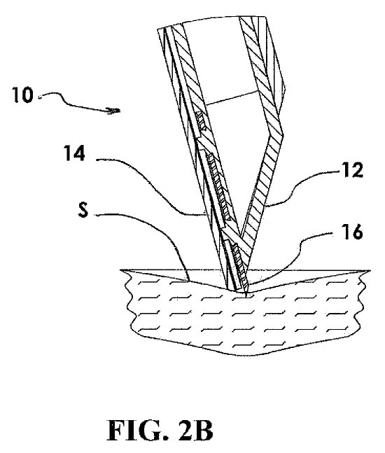

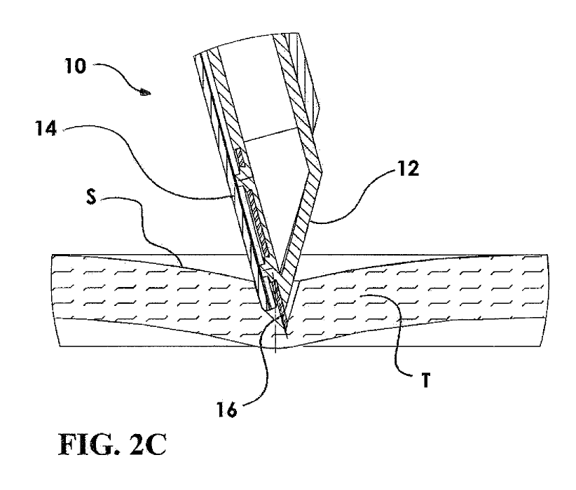

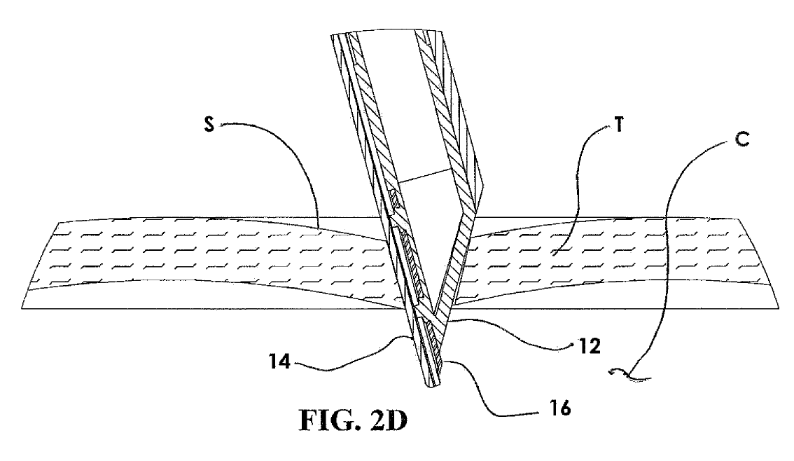

As illustrated in FIGS. 2A-2D, and as described above herein, the present invention also includes a method of positioning and using an optical safety trocar 10. For example, an embodiment of a method of using an optical safety trocar 10 includes installing an optical tip 12 onto a distal end of a tubular trocar 20, proximate a blade 16, and adapting a shield 14 for protectively surrounding blade 16 while deployed. Pressing deployed shield 14 against skin S of a patient, as shown in FIG. 2A, in a first step, wherein continued pressure partially retracts shield 14 to partially expose blade 16, as shown in FIG. 2B. Upon full exposure of blade 16 during full retraction of shield 14, as shown in FIG. 2C, skin S and tissue T are cut thereby, with continued visualization of the process enabled through optical tip 12. Following penetration into the body cavity C, as shown in FIG. 2D, shield 14 redeploys and prevents further cutting by blade 16 via shield length 74, even before passage of length 76 into body cavity C.

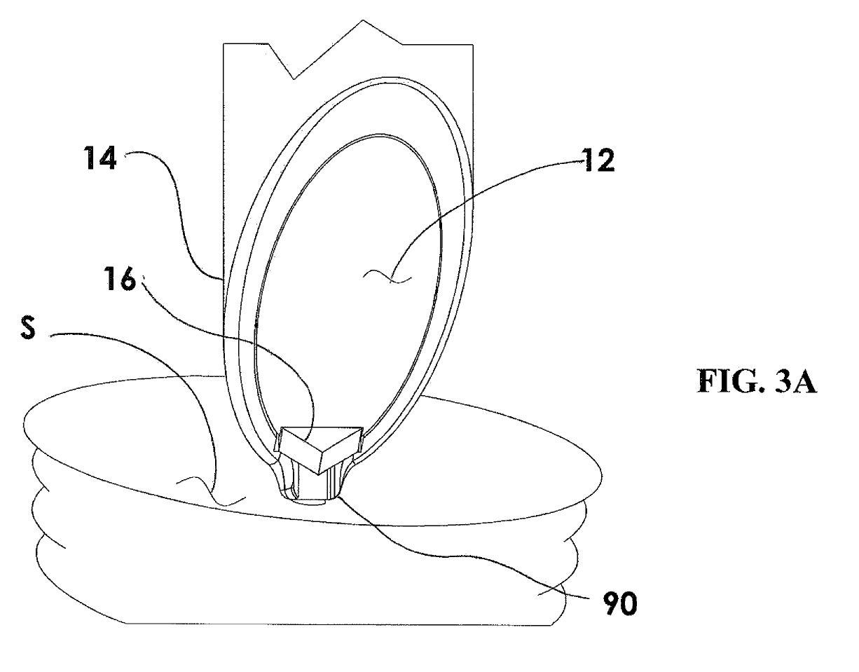

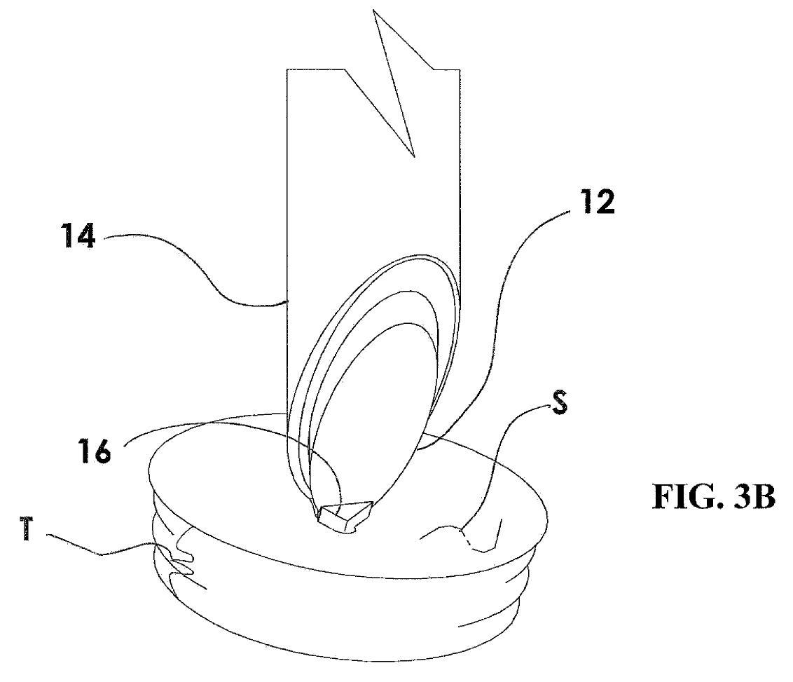

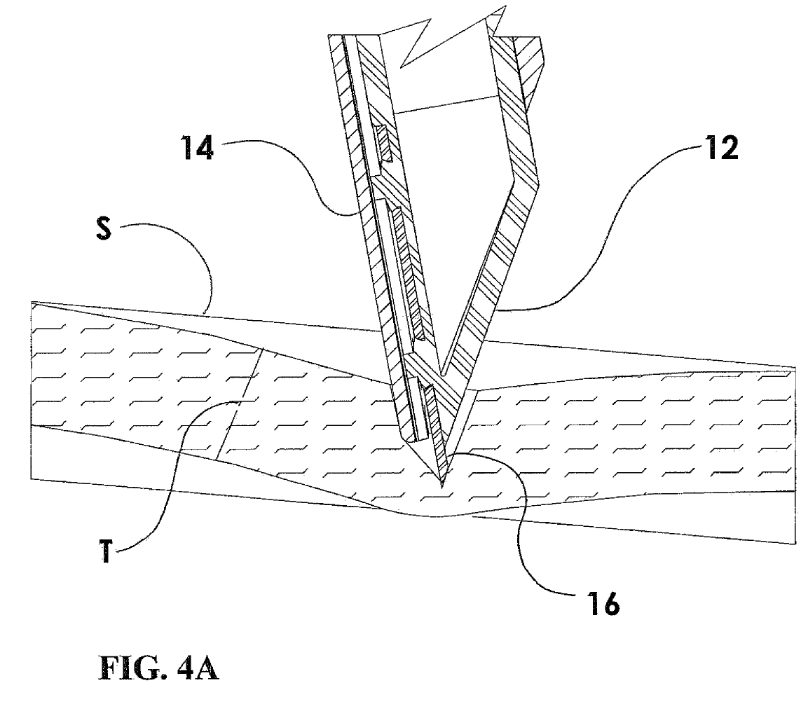

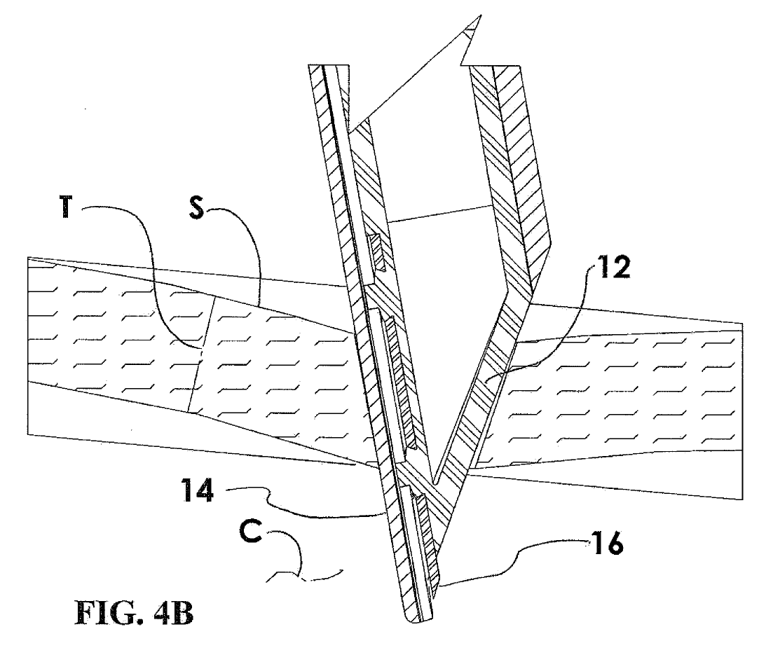

Again, another method of use is also shown in FIGS. 3A-4B, wherein shield 14 further comprises blunt nose 90, such that blunt nose 90 facilitates advance engagement of shield 14 upon contact with skin S in order to initiate retraction of shield 14 with delivery of minimized force. As shown in FIG. 3B, initialized retraction of shield 14 begins to expose blade 16 to facilitate creation of an incision. As depicted in FIG. 4A, blade 16 cuts through skin S while shield 14 is retracted, again wherein visualization is facilitated via optical tip 12. With further reference to FIG. 4B, shield 14 redeploys proximate blade 16 upon entry into body cavity C, wherein viewing continues to be facilitated via optical tip 12.

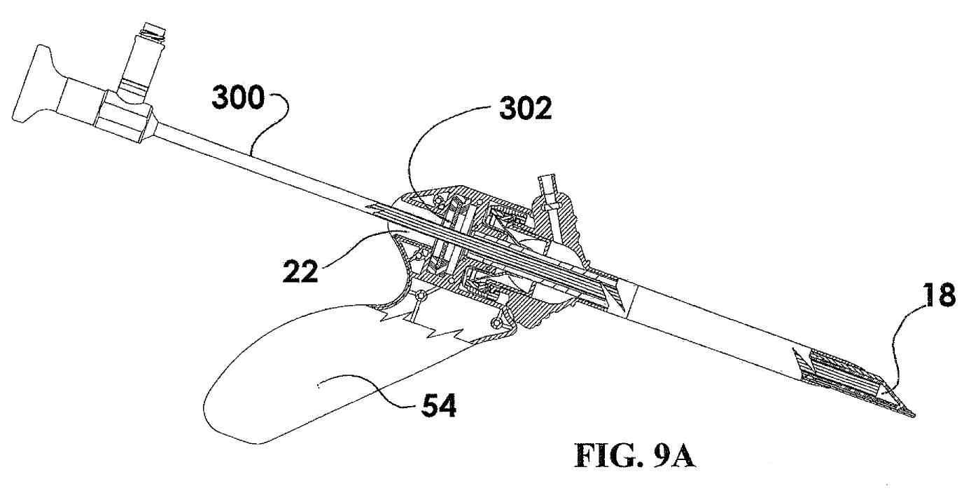

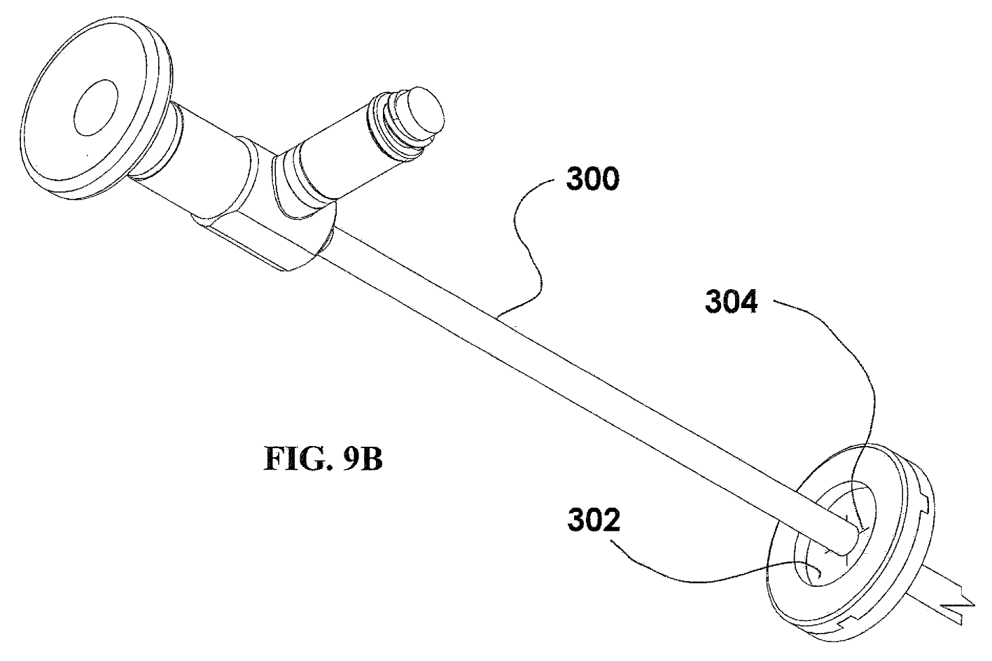

As further depicted in FIG. 9A, an endoscope, 300, is extended into proximate end 22 of channel 18, wherein endoscope 300 is adapted with elastomeric diaphragm 302. Elastomeric diaphragm 302 functions to stabilize and support scope 300, wherein plurality of slits 304 prevent sealing while supporting scope 300, and further facilitate entry of larger size scopes.

Having thus described exemplary embodiments of the present invention, it should be noted by those skilled in the art that the within disclosures are exemplary only, and that various other alternatives, adaptations, and modifications may be made within the scope of the present invention. Accordingly, the present invention is not limited to the specific embodiments illustrated herein, but is limited only by the following claims.

We claim:

1. A trocar, comprising:
a cannula assembly having a cannula;
an obturator composed of:
a longitudinal bore between a proximal end and a distal end, said longitudinal bore being configured to receive at least a portion of an endoscope;
a distal tip which facilitates the transmission of an image;
a means for penetrating body tissue incorporated into said distal tip; and
a shield slidably mounted to the trocar body.

2. The trocar of claim 1, wherein said means for penetrating body tissue is a blade.

3. The trocar of claim 1, wherein said means for penetrating body tissue is a bladeless tip.

4. The trocar of claim 1, wherein the shield is biased in a deployed position.

5. The trocar of claim 1, wherein said trocar further comprises a shield stop.

6. A surgical tool, comprising:
a handle having an accessway adapted for passage of a medical instrument therethrough;
an elastomeric diaphragm within said handle, said diaphragm adapted for stabilizing the medical instrument;
a tubular trocar having an accessway adapted for receiving a visualization implement, having a springedly-engaged shield, and having a visualization tip; and
a cannula attached to said handle and adapted to receive said tubular trocar,

7. The surgical tool of claim 6, wherein said accessway of said tubular trocar is adapted for receiving an endocscope.

8. The surgical tool of claim 6, wherein said visualization tip is optically transparent.

9. The surgical tool of claim 6, wherein said tubular trocar has a distal end, wherein said visualization tip and said springedly-engaged shield are proximate said distal end, wherein the profile of said distal end is angular, wherein said visualization tip is angularly defined within said angular profile of said distal end of said trocar, and wherein said shield is generally tubular, with a first axial length and a second axial length different than said first axial length, thereby defining an angular profile for said shield proximate said distal end of said trocar.

10. The surgical tool of claim 6, further comprising a blade.

11. The surgical tool of claim 9, further comprising a blade, wherein said blade is carried proximate a portion of said shield with greatest axial length.

12. The surgical tool of claim 11, wherein said axial length of said shield extends further than said blade relative to said distal end of said trocar according to a springed bias first position, wherein said shield is adapted for slidable movement relative to said trocar, and wherein slidable movement of said shield to a second position in a direction away from said distal end of said trocar changes the relative positioning of said shield and said blade, wherein said blade extends further than said shield relative to said distal end of said trocar.

13. The surgical tool of claim 6, wherein said visualization tip and said shield define a distal end of said trocar that is formed at a preselected angle relative to a plane perpendicular to an axis of said trocar.

14. The surgical tool of claim 6, wherein said visualization tip has a blunt nose extension.

15. A method of using a trocar, comprising the steps of:
obtaining safety trocar 10 comprising an instrument channel, an optical tip carried on a distal end proximate a blade, and a shield positioned to surround said blade, said shield adapted for springedly-biased positioning and said optical tip, said blade and said shield defining an angular profile for said distal end of said safety trocar;
pressing said shield against the skin of the patient, thereby causing said shield to slidably relocate away from said distal end of said safety trocar;
exposing said blade;
utilizing said blade to cut the skin and tissue of the patient while visibly observing the action of said blade via said instrument channel;
allowing said blade to pass into a body cavity while visibly observing the action of said blade via said instrument channel;
allowing at least a portion of said shield to pass into the body cavity, thereby causing said shield to slidably relocate toward said distal end of said safety trocar while visibly observing the action of said blade via said instrument channel.

16. The method of claim 15, further comprising the steps of:
obtaining a cannula and inserting said safety trocar therein prior to pressing said shield against the skin of the patient;
allowing said cannula to pass into the body cavity while visibly observing the action of said blade via said instrument channel;
withdrawing said safety trocar from the body cavity.

17. The surgical tool of claim 7, wherein said endoscope further comprises an elastomeric diaphragm.

18. The surgical tool of claim 17, wherein said elastomeric diaphragm further comprises a plurality of radial slits.
